(19) United States
(12) Patent Application Publication
(10) Pub. No.: US 2025/0360050 A1
(43) Pub. Date: Nov. 27, 2025

(54) ULTRASOUND-GUIDED BIOPSY AUXILIARY DEVICE AND ULTRASOUND PROBE INCLUDING THE SAME

(71) Applicant: HUKUI BIOTECHNOLOGY CO., LTD., Taipei City (TW)

(72) Inventors: SHIH-CHE LO, Taipei City (TW); CHUN-CING CHEN, Taipei City (TW)

(73) Assignee: HUKUI BIOTECHNOLOGY CO., LTD., Taipei City (TW)

(21) Appl. No.: 19/214,063

(22) Filed: May 21, 2025

Related U.S. Application Data
(60) Provisional application No. 63/651,412, filed on May 24, 2024.

(30) Foreign Application Priority Data
Jul. 26, 2024 (TW) 113127687

(51) Int. Cl. A61H 23/02; A61M 25/09; A61N 7/00

(52) U.S. Cl. CPC A61H 23/0245; A61H 2201/165; A61M 25/09; A61N 7/00

(57) ABSTRACT

An ultrasound-guided biopsy auxiliary device includes a body, the body includes a sampling conduit penetrating through the body; an injection port located on a surface of the body and in fluid communication with the sampling conduit; and a plurality of positioning marks located in the body, wherein the plurality of positioning marks are arranged along at least two directions perpendicular to each other, and in either direction, spacings between the positioning marks are in the same size. The ultrasound-guided biopsy auxiliary device is suitable for assisting the ultrasound-guided biopsy of a human body.

### CROSS-REFERENCE TO RELATED APPLICATION

This non-provisional application claims priority under 35 U.S.C. § 119(a) on Patent Application No(s). 113127687 filed in Taiwan, R.O.C. on Jul. 26, 2024, the entire contents of which are hereby incorporated by reference.

This non-provisional application claims priority under 35 U.S.C. § 119(e) on US provisional Patent Application No(s). 63/651,412 filed on May 24, 2024, the entire contents of which are hereby incorporated by reference.

### BACKGROUND OF THE INVENTION

### 1. Field of the Invention

The present disclosure relates to an ultrasound-guided biopsy auxiliary device, in particular to an ultrasound-guided biopsy auxiliary device including a sampling conduit, an injection port and a plurality of positioning marks. The present disclosure also relates to an ultrasound probe including the ultrasound-guided biopsy auxiliary device.

### 2. Description of the Related Art

Ultrasound imaging technology is widely used in the diagnoses conducted on human bodies. The ultrasound generator inside the ultrasound probe is composed of many small units, and interfering within a certain distance exists among the ultrasonic waves generated by these small units, resulting in poor imaging quality of ultrasound images. The zone where interferences occur is generally referred to as the “Near Field”. Beyond a certain distance, the ultrasonic waves generated by these small units do not interfere; and beyond the distance, the ultrasonic energy generated by these small units is evenly distributed, and the waveform is similar to that generated by a single point source. As a result, the imaging quality of the ultrasound image is good beyond the distance. In contrast to the near field, the zone where no interference occurs is generally referred to as the “Far Field”.

Because of the above characteristics, a specific distance must be maintained between the ultrasound probe and the target zone in which image is to be formed, so that the target zone is located in the far field to obtain good imaging quality. However, due to the differences in the depths of different organs in the human body, usually it is difficult to make a single probe suitable for diagnoses conducted on all organs in the human body.

In addition, ultrasound imaging technology has also been widely used to guide needles for human tissue sampling. However, conventionally, when using ultrasound imaging technology to guide needles for human tissue sampling, the penetration point of the needle and the position of the ultrasound probe must be determined completely based on the experiences of the physician, which makes it with relatively high uncertainty; and it is difficult to ensure that every time the tissue of the target lesion can be accurately sampled.

### BRIEF SUMMARY OF THE INVENTION

There is still room for improvement in conventional ultrasound imaging technology. Therefore, one object of the present disclosure is to provide an ultrasound-guided biopsy auxiliary device that can adjust the distance between the ultrasound probe and the target zone to be imaged, so as to improve the imaging quality and guide the needle for human tissue sampling, thereby improving the sampling accuracy.

In order to achieve the above object and other objects, the present disclosure provides an ultrasound-guided biopsy auxiliary device including:
a body including:
a sampling conduit penetrating through the body;
an injection port located on a surface of the body and in fluid communication with the sampling conduit; and
a plurality of positioning marks located in the body, wherein the positioning marks are arranged along at least two directions perpendicular to each other, and in either direction, spacings between the positioning marks are in the same size.

In an embodiment of the present disclosure, the sampling conduit may have a tapered structure.

In an embodiment of the present disclosure, the body is made of silicone, polyurethane or rubber.

In an embodiment of the present disclosure, a thickness of the body is between 5 mm and 50 mm.

In order to achieve the above object and other objects, the present disclosure also provides an ultrasound probe including the ultrasound-guided biopsy auxiliary device described as above at a front end.

In an embodiment of the present disclosure, the ultrasound-guided biopsy auxiliary device is integrally fastened to the front end of the ultrasound probe.

In an embodiment of the present disclosure, the ultrasound-guided biopsy auxiliary device is detachably mounted to the front end of the ultrasound probe.

In an embodiment of the present disclosure, the ultrasound probe may include a capacitive micromachined ultrasonic transducer (CMUT) or a piezoelectric micromachined ultrasonic transducer (PMUT).

By providing the sampling conduit, the injection port and the plurality of positioning marks, it is possible for the ultrasound-guided biopsy auxiliary device and the ultrasound probe of the disclosure to improve the imaging quality and the sampling accuracy.

### BRIEF DESCRIPTION OF THE DRAWINGS

For clarity, the thickness or size of each layer in the drawings is enlarged, omitted or outlined. Therefore, the dimensions of each element do not fully reflect their true dimensions.

FIG. 1 is a schematic sectional view of an ultrasound-guided biopsy auxiliary device of the embodiment 1 according to the present disclosure.

FIG. 2 is a schematic sectional view of the ultrasound-guided biopsy auxiliary device of the embodiment 1 according to the present disclosure in a state of use.

FIG. 3 is a schematic sectional view of an ultrasound probe of the embodiment 2 according to the present disclosure.

### DETAILED DESCRIPTION OF THE INVENTION

The objectives, characteristics and technical effects of the present disclosure can be fully understood by means of describing of the present disclosure in detail based on the following specific embodiments as provided below.

### Embodiment 1

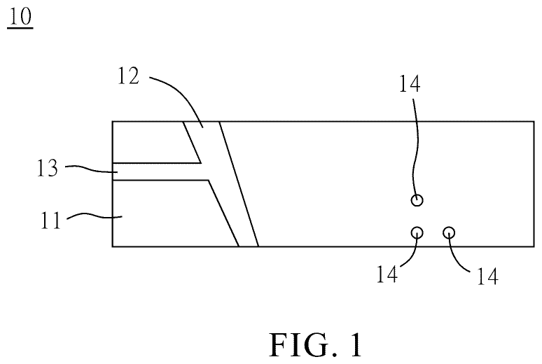

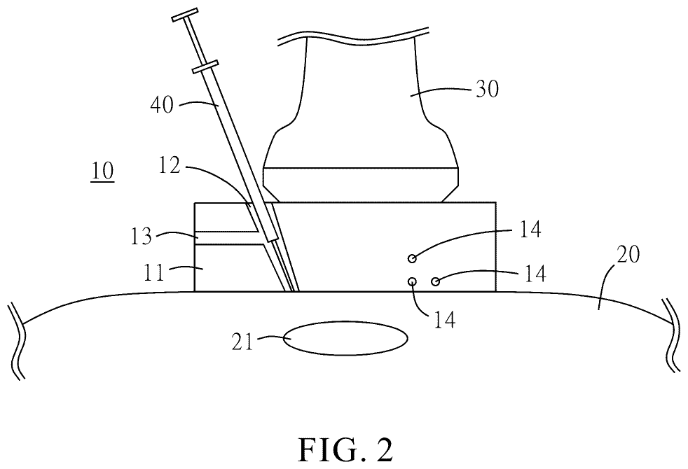

An ultrasound-guided biopsy auxiliary device of the embodiment 1 according to the present disclosure is shown in FIG. 1 and FIG. 2.

The ultrasound-guided biopsy auxiliary device 10 of the embodiment according to the present disclosure includes a body 11. For example, the body 11 is preferably made of an elastomer such as silicone, polyurethane or rubber; but the present disclosure is not limited thereto.

The body 11 includes a sampling conduit 12 penetrating through the body 11. In the embodiment, the sampling conduit 12 has a tapered structure; however, as long as a sampling device 40 can be properly guided, the present disclosure is not limited thereto.

The body 11 also includes an injection port 13 located on a surface of the body 11 and in fluid communication with the sampling conduit.

The body 11 also includes a plurality of positioning marks 14 located in the body 11, wherein the positioning marks 14 are arranged along at least two directions perpendicular to each other, and in either direction, spacings between the positioning marks 14 are in the same size. Preferably, there are considerable differences in the ultrasound conduction properties between the plurality of positioning marks 14 and the body 11, so that the plurality of positioning marks 14 can be clearly imaged by ultrasound. For example, a material having a density significantly higher than that of the body 11 may be selected to form the plurality of positioning marks 14.

As shown in FIG. 2, when using the ultrasound-guided biopsy auxiliary device 10 of the embodiment according to the present disclosure, a lower surface of the device is attached to the human body 20, whereby flattening the surface of the human body 20 to enhance the convenience of operation. For example, when sampling the thyroid gland located in the larynx, the ultrasound-guided biopsy auxiliary device 10 of the embodiment according to the present disclosure can be used to flatten the larynx, thereby preventing the ultrasound probe 30 from undesired sliding due to the uneven surface of the larynx during operation.

As shown in FIG. 2, when the ultrasound-guided biopsy auxiliary device 10 of the embodiment according to the present disclosure in use, the ultrasound probe 30 is fitted to an upper surface of the ultrasound-guided biopsy auxiliary device 10. The sampling device 40 can be in contact with the human body 20 through the sampling conduit 12, and then penetrates the human body 20 to sample at a lesion 21.

As shown in FIG. 2, the ultrasound-guided biopsy auxiliary device 10 of the embodiment according to the present disclosure can keep the ultrasound probe 30 at a certain distance from the lesion 21, so as to avoid poor imaging caused by interference phenomenon when the lesion 21 is located in a relatively superficial area of the human body (for example, the thyroid gland in the throat), the distance between the ultrasound probe 30 and the lesion 21 is too close and thus the lesion 21 is in the near field.

In use, a liquid such as normal saline is injected by means of the injection port 13 to fill the inside of the sampling conduit 12, so as to prevent the ultrasound from being reflected by the air in the sampling conduit 12 when the ultrasound passes through the sampling conduit 12, thereby affecting the transmission efficiency and thus resulting in poor imaging quality; and in addition, the normal saline also ensures that the inside of the sampling conduit 12 is in a sterile environment, so as to avoid possible contamination during the process of sampling.

At the time of sampling, the operator can concurrently observe the plurality of positioning marks 14 and the lesion 21 in the ultrasound images, in order to determine the relative distance between the plurality of positioning marks 14 and the lesion 21. Since the relative distance between the plurality of positioning marks 14 and the sampling conduit 12 is fixed, the relative distance between the sampling conduit 12 and the lesion 21 can be determined based on the relative distance between the plurality of positioning marks 14 and the lesion 21, and thus, the operator can determine whether or not an opening of the sampling conduit 12 on the lower surface of the body 11 is aligned with the lesion 21, thereby determining whether it is suitable for sampling by means of the sampling device 40; or it is necessary to further move the ultrasound-guided biopsy auxiliary device 10 to make the opening of the sampling conduit 12 on the lower surface of the body 11 aligned with the lesion 21, thereby improving the sampling accuracy. In addition, during the process of sampling through the sampling device 40, by providing the sampling conduit 12, before a needle of the sampling device 40 penetrates a tissue of the human body 20, the image of the needle of the sampling device 40 and the lesion 21 to be sampled can be seen simultaneously in the ultrasound image, thereby reducing position deviation or the pain caused to the patient because of the need for readjustment due to position misalignment. It can even be seen in advance whether there are any blood vessels or tissues that need to be avoided in the entry path of the needle of the sampling device 40, so as to avoid accidental damage to the human body 20.

For example, the ultrasound-guided biopsy auxiliary device 10 of the embodiment according to the present disclosure may be applied to thyroid biopsy, breast ultrasound-guided biopsy or liver pus biopsy; however, the present disclosure is not limited thereto.

In one preferred embodiment of the present disclosure, the body 11 has a thickness between 5 mm and 50 mm, so as to facilitate the lesion 21 to be sampled being in the far field with preferable imaging quality; however, the present disclosure is not limited thereto, and a person having ordinary skill in the art can freely adjust the thickness of the body 11 according to demand.

### Embodiment 2

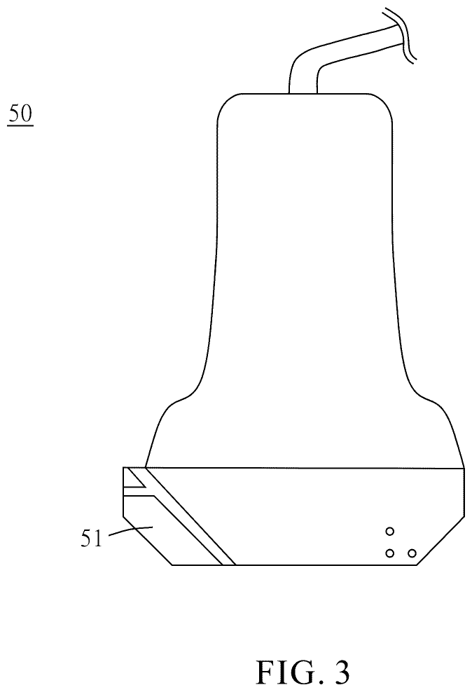

An ultrasound probe of the embodiment 2 according to the present disclosure is shown in FIG. 3.

The ultrasound probe 50 of the embodiment 2 according to the present disclosure includes the ultrasound-guided biopsy auxiliary device 51 as described in the embodiment 1 at a front end. For example, the ultrasound probe 50 includes a capacitive micromachined ultrasonic transducer (CMUT) or a piezoelectric micromachined ultrasonic transducer (PMUT); however, the present disclosure is not limited thereto.

In the ultrasound probe 50 of the embodiment 2 according to the present disclosure, the ultrasound-guided biopsy auxiliary device 51 as described in the embodiment 1 is integrated into the front end of the probe, and thus, the imaging quality can be improved and the sampling accuracy can be improved by providing the sampling conduit, the injection port and the plurality of positioning marks of the ultrasound-guided biopsy auxiliary device 51.

In the ultrasound probe 50 of the embodiment 2 according to the present disclosure, the ultrasound-guided biopsy auxiliary device 51 is integrally mounted to the front end of the ultrasound probe 50; however, the present disclosure is not limited thereto. In another embodiment, the ultrasound-guided biopsy auxiliary device 51 is detachably provided at the front end of the ultrasound probe 50, and thus, when there is no need to sample and the lesion is located in a deeper part of the human body, the operator can choose to use the ultrasound probe 50 in absence of the ultrasound-guided biopsy auxiliary device 51, so as to increase the convenience of use. In another state of use, by detachably providing the ultrasound-guided biopsy auxiliary device 51 at the front end of the ultrasound probe 50, the operator can replace different auxiliary devices based on actual needs, so as to increase the diversity of use.

While the present invention has been described by means of preferred embodiments, those skilled in the art should understand that the foregoing embodiments is merely for describing the invention, and these embodiments should not be considered to be limiting the scope of the claimed invention. It should be noted that all modifications and replacements equivalent to these embodiments shall be falling within the scope of the claimed invention. Therefore, the scope of the invention is defined by the appended claims.

While the present disclosure has been described by means of specific embodiments, numerous modifications and variations could be made thereto by those skilled in the art without departing from the scope and spirit of the present disclosure set forth in the claims.

What is claimed is:

1. An ultrasound-guided biopsy auxiliary device comprising:
a body comprising:
a sampling conduit penetrating through the body;
an injection port located on a surface of the body and in fluid communication with the sampling conduit; and
a plurality of positioning marks located in the body, wherein the positioning marks are provided along at least two directions perpendicular to each other, and in either direction, spacings between the positioning marks are in the same size.

2. The ultrasound-guided biopsy auxiliary device according to claim 1, wherein the sampling conduit has a tapered structure.

3. The ultrasound-guided biopsy auxiliary device according to claim 1, wherein the body is made of silicone, polyurethane or rubber.

4. The ultrasound-guided biopsy auxiliary device according to claim 1, wherein the body has a thickness between 5 mm and 50 mm.

5. An ultrasound probe comprising the ultrasound-guided biopsy auxiliary device according to claim 1 at a front end.

6. The ultrasound probe according to claim 5, wherein the ultrasound-guided biopsy auxiliary device is integrally mounted to the front end of the ultrasound probe.

7. The ultrasound probe according to claim 5, wherein the ultrasound-guided biopsy auxiliary device is detachably provided at the front end of the ultrasound probe.

8. The ultrasound probe according to claim 5, wherein the ultrasound probe comprises a capacitive micromachined ultrasonic transducer (CMUT) or a piezoelectric micromachined ultrasonic transducer (PMUT).
